(19) United States
(12) Patent Application Publication
(10) Pub. No.: US 2025/0283787 A1
(43) Pub. Date: Sep. 11, 2025

(54) Dissolved Gas Analysis System and Method

(71) Applicant: AVO Multi-Amp Corporation d/b/a Megger, Dallas, TX (US)

(72) Inventors: Renyan Qin, Phoenixville, PA (US); Thomas Michael Liptack, Phoenixville, PA (US); Jeffrey Dean Benach, Phoenixville, PA (US); Robert Earl Collins, Spring Mount, PA (US)

(21) Appl. No.: 18/596,307

(22) Filed: Mar. 5, 2024

(51) Int. Cl. G01N 1/22; G01N 1/14; G01N 1/40; G01N 33/28; H01F 27/40

(52) U.S. Cl. CPC G01N 1/2202; G01N 1/14; G01N 1/4005; G01N 33/2841; H01F 27/402; H01F 2027/404

(57) ABSTRACT

A system for gas analysis is provided. The system includes a gas extraction assembly having an extraction chamber having an upper end, a lower end, and an interior space. The gas extraction assembly further having one or more retainers positioned within the interior space of the extraction chamber. The gas extraction assembly further having gas permeable membrane retained within the interior space of the extraction chamber by the one or more retainers, the gas permeable membrane disposed in S-shaped curves extending from adjacent the upper end to adjacent the lower end within the interior space of the extraction chamber.

### BACKGROUND

Electrical power systems including components such as transformers are known to degrade and fail due to aging, thermal and electrical stresses, in which cases the breakdown of insulation material, for example, oil and paper occur. The breakdown of the oil generates certain gases commonly referred to as “fault gases” dissolved in it. Such fault gases may include hydrogen (H₂), carbon monoxide (CO), carbon dioxide (CO₂), methane (CH₄), acetylene (C₂H₂), ethylene (C₂H₄), and ethane (C₂H₆). It is well known in the industry that the amount and nature of these fault gases can be used to identify the type and severity of the fault in the transformer. Therefore, during operation, these dissolved gases may be analyzed or monitored continuously and in real time to evaluate the operational status of the transformer.

### SUMMARY

Described herein is a system and method for online dissolved gas analysis. According to one embodiment, a system for gas analysis is provided. The system includes a gas extraction assembly having an extraction chamber having an upper end, a lower end, and an interior space. The gas extraction assembly further having one or more retainers positioned within the interior space of the extraction chamber. The gas extraction assembly further having gas permeable material retained within the interior space of the extraction chamber by the one or more retainers, the gas permeable material disposed in S-shaped curves extending from adjacent the upper end to adjacent the lower end within the interior space of the extraction chamber.

According to another embodiment, a gas analysis system is provided that includes a transformer, a gas extraction assembly, a manifold, and an attachment system. The transformer includes an electrical winding and a fluid system comprising insulating fluid. The gas extraction assembly includes an extraction chamber and gas permeable material retained within the extraction chamber. The manifold has a first interface and a second interface, the second interface in fluid communication with the gas extraction assembly, and one or more channels to communicate the fluid between the first and second interfaces. The attachment system includes an adapter pipe, a first interface, and a second interface. The adapter pipe has a first end, a second end, an elongate portion extending from the first end towards the second end, and a bend portion provided adjacent the elongate portion and extending to adjacent the second end. The first interface adjacent the first end of the adapter pipe in fluid communication with the transformer, and the second interface adjacent the second end of the adapter pipe in fluid communication with the first interface of the manifold.

According to yet another embodiment, a method analyzing dissolved gases in a fluid of a transformer is provided. The method includes providing an adapter pipe having a first end and a second end, the first end in fluid communication with the fluid of the transformer, the second end in fluid communication with a gas analysis system, the adapter pipe having an extension tube provided within the adapter pipe and extending within the adapter pipe between the first end and the second end of the adapter pipe. The method includes supplying the fluid through the extension tube from a first end of the extension tube adjacent the first end of the adapter pipe to a second end of the extension tube adjacent the second end of the adapter pipe, the second end of the extension tube coupled to communicate with a first port of the gas analysis system. The method includes communicating the fluid from the second end of the extension tube, via the first port, into an extraction chamber of the gas analysis system, the extraction chamber having gas permeable material disposed therein. The method includes obtaining the dissolved gas via the gas permeable material, analyzing characteristics of the dissolved gas, and communicating the fluid from the extraction chamber to a second port of the gas analysis system. The method also includes returning the fluid, via the second port, to the second end of the adapter pipe, the second port adjacent the first port such that the supply fluid is supplied adjacent the first end of the adapter pipe and return fluid is returned adjacent the second end of the adapter pipe or via other connection.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 is a front perspective view of a gas analysis system according to one embodiment of the present disclosure.

FIG. 2 is a side view of the gas analysis system according to an embodiment of the present disclosure.

FIG. 3A is a top perspective view of a retainer of the gas analysis system according to an embodiment of the present disclosure.

FIG. 3B is partial perspective view of the retainer of the gas analysis system shown in place according to an embodiment of the present disclosure.

FIG. 3C is cross-sectional view of the retainer of the gas analysis system shown in place according to an embodiment of the present disclosure.

FIG. 3D is a side perspective view of a supply conduit of the gas analysis system show with the retainers removed.

FIG. 4 is a layout view of gas permeable material of the gas analysis system according to an embodiment of the present disclosure.

FIG. 5 is a top view of the gas permeable material of the gas analysis system according to an embodiment of the present disclosure.

FIG. 6 is a side perspective view of the gas permeable material of the gas analysis system according to an embodiment of the present disclosure.

FIG. 7 illustrates a support member of the gas analysis system according to an embodiment of the present disclosure.

FIG. 8 is a back view of the gas analysis system according to an embodiment of the present disclosure.

FIG. 9 is a perspective view of an attachment system and the gas analysis system according to an embodiment of the present disclosure.

FIG. 10A is a side cut-away view of the attachment system and the gas analysis system according to another embodiment of the present disclosure.

FIG. 10B is an exploded side cut-away view of a portion of the attachment system and the gas analysis system according to another embodiment of the present disclosure.

FIG. 11 is an exploded side cut-away of a portion of the attachment system illustrated in FIG. 10 taken along “C” according to an embodiment of the present disclosure.

FIG. 12 is a side view of another embodiment of the attachment system and the gas analysis system according to an embodiment of the present disclosure.

FIG. 13 is a bottom perspective view of an embodiment of the attachment system and the gas analysis system according to the present disclosure.

FIG. 14 is a top perspective view of the attachment system and the gas analysis system illustrated in FIG. 13 according to the present disclosure.

FIG. 15 is a side view of a remote attachment system and the gas analysis system according to an embodiment of the present disclosure.

FIG. 16 is a front perspective view of a mount adapter of the remote attachment system according to an embodiment of the present disclosure.

FIG. 17 is a back perspective view of the mount adapter of the remote attachment system according to an embodiment of the present disclosure.

FIG. 18 is a side cut-away view of the mount adapter of the remote attachment system according to an embodiment of the present disclosure.

FIG. 19 is a flow chart of a method analyzing dissolved gases in a fluid of a transformer according to one embodiment of the present disclosure.

FIG. 20 is a diagram of an embodiment of a system suitable for implementing various embodiments of the present disclosure.

### DETAILED DESCRIPTION

Described herein is a system and method for dissolved gas analysis. As mentioned above, during operation of transformers, fluid used to cool and insulate the transformer may develop dissolved gas. Analysis of the dissolved gas can provide information that can be used, for example, as an indicator of the operational status of the transformer.

In order to extract the dissolved gas from the fluid, gas permeable material may be subjected to the transformer fluid to obtain the dissolved gas for analysis. The present disclosure has observed that greater surface exposure of the gas permeable material to the fluid yields increased opportunity for gas to pass through the gas permeable material for analysis. Briefly, the present disclosure provides an extraction chamber, one or more retainers positioned within the extraction the chamber, and gas permeable material retained within the interior space of the extraction chamber by the one or more retainers. The gas permeable material is disposed in S-shaped curves extending from adjacent the upper end to adjacent the lower end within the interior space of the extraction chamber. In addition, transformers are not universal or standardized in all aspects of design and location. Accordingly, the present disclosure provides a manifold that provides for optional configurations for attachment of the present system to access the transformer fluids via attachment systems also disclosed herein. These and other features and advantages are disclosed in greater detail herein.

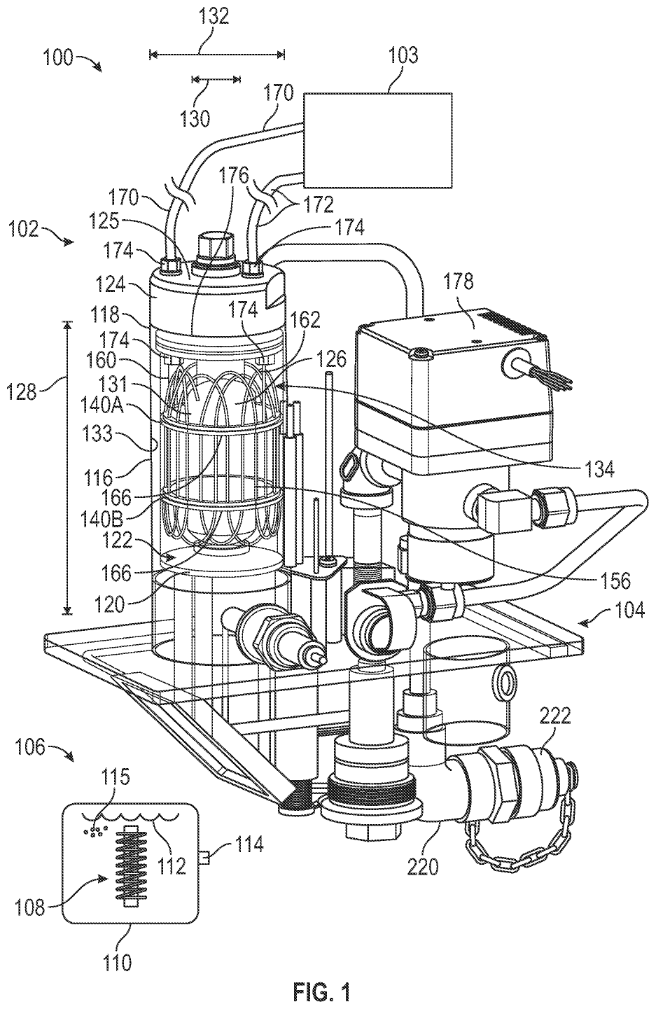

FIG. 1 is a front perspective view illustrating of an embodiment of a gas analysis system 100. The gas analysis system 100 includes a gas extraction assembly 102, a gas analyzer 103, and a manifold 104. The gas analysis system 100 may be attached or in fluid communication with an electrical power system, such as a transformer 106. The transformer 106, in this embodiment, may be a core-type, high voltage, three-phase transformer 106, although other types of transformers or electrical systems may be used. The transformer 106 includes electrical windings 108 disposed in a housing 110. Fluid 112, such as dielectric fluid, may be provided in the housing 110 for cooling and insulating the transformer 106 components. As discussed above, dissolved gas 115 may develop in the fluid 112 during operation for various reasons. Accordingly, the transformer 106 includes a valve 114 that is in fluid communication with the fluid 112 in the housing 110 and provides ready access to fluid 112. In some embodiments, the valve 114 may include any component or fitting to enable access to the fluids 112 within the transformer 106.

The gas analyzer 103 includes various gas sensor modules, including but not limited to, the gas sensors based on infrared absorption, thermal conductivity, and electrochemistry, for conducting analysis of the gas collected according to the present system. The gas analyzer 103 may perform any number or manner of gas analysis techniques necessary or desirable to indicate the operational status of the fluid 112 and/or the transformer 106.

The gas extraction assembly 102 includes an extraction chamber 116, which in this embodiment may be a cylindrically shaped cover tube or pipe, although other configurations of the extraction chamber 116 may be used. The extraction chamber 116 extends from an upper end 118 to a lower end 120. The extraction chamber 116 may be attached to the manifold 104 adjacent the lower end 120 and include a cap 124 or cover coupled adjacent the upper end 118. The extraction chamber 116 may be attached to the manifold 104 and cap 124 via various attachment systems, such as, but not limited to, by snap rings, threaded attachment, or other attachment systems. The extraction chamber 116 coupling to the manifold 104 adjacent the lower end 120 and cap 124 adjacent the upper end 118 define an interior space 122 within the extraction chamber 116.

A supply conduit 126, which may be a pipe or tube, is centrally located in the interior space 122 of the extraction chamber 116. The supply conduit 126 may generally be about a length 128 or height of the extraction chamber 116. However, the supply conduct 126 has a smaller outer diameter 130, as defined by an outer wall 131 of the supply conduit 126, than that of an inner diameter 132 of the extraction chamber 116, as defined by an inner wall 133 of the extraction chamber 116. The area between the inner diameter 132 of the extraction chamber 116 and outer diameter 130 of the supply conduit 126 defines a gas extraction area 134 within the interior space 122 of the extraction chamber 116.

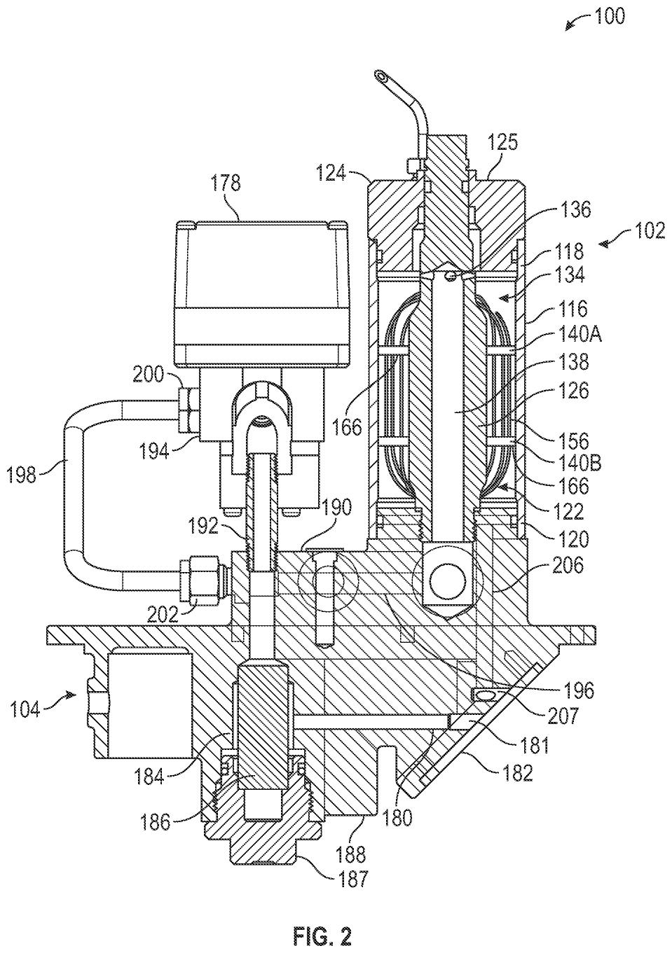

Referring also to FIG. 2, a side view of the gas analysis system 100 is illustrated. The supply conduit 126 is similarly attached to the manifold 104 adjacent the lower end 120 and the cap 124 adjacent the upper end 118. In the present embodiment the supply conduit 126 may extend through an upper side 125 of the cap 124. The extraction chamber 116 may be attached to the manifold 104 and cap 124 via various attachment systems, such as, but not limited to, by snap rings, threaded attachment, or other attachment systems. As can be seen in FIG. 2, the supply conduit 126 includes multiple through holes 136 that allow fluid to be communicated from within a passageway 138 through the supply conduit 126 into the gas extraction area 134. In the present embodiment, four cross-drilled through holes 136 are provided, however fewer or more openings may be provided and otherwise located in other embodiments.

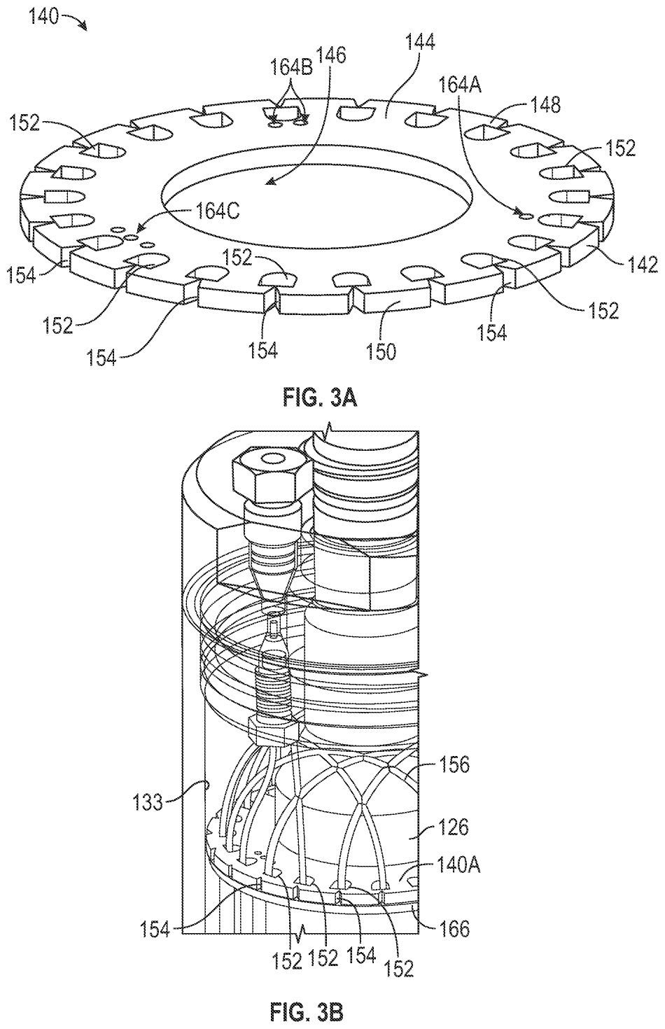

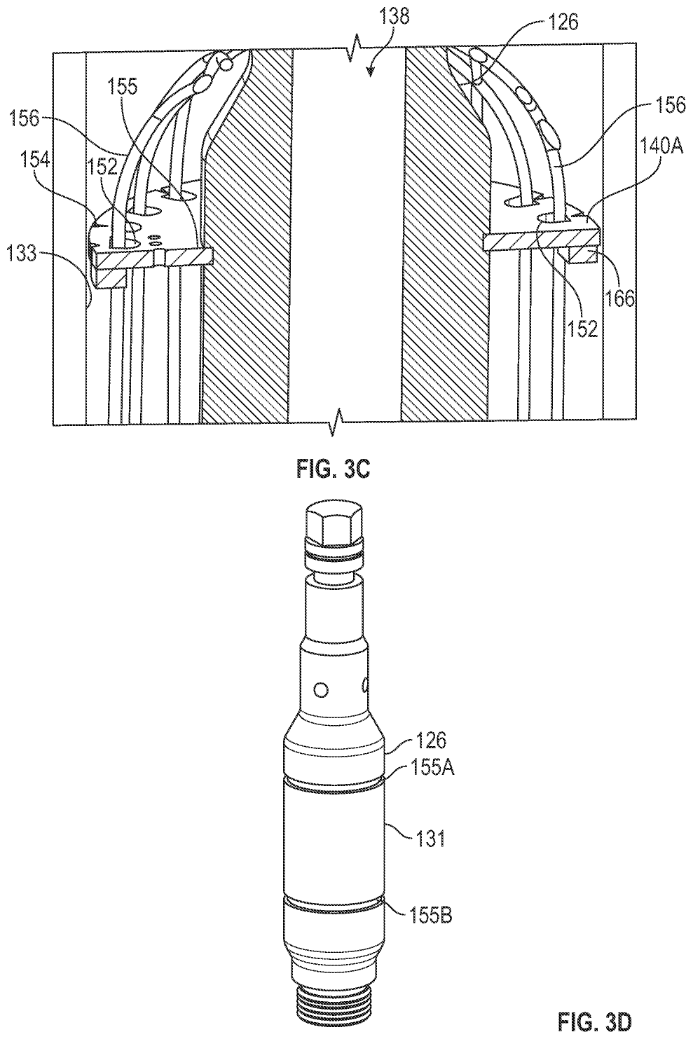

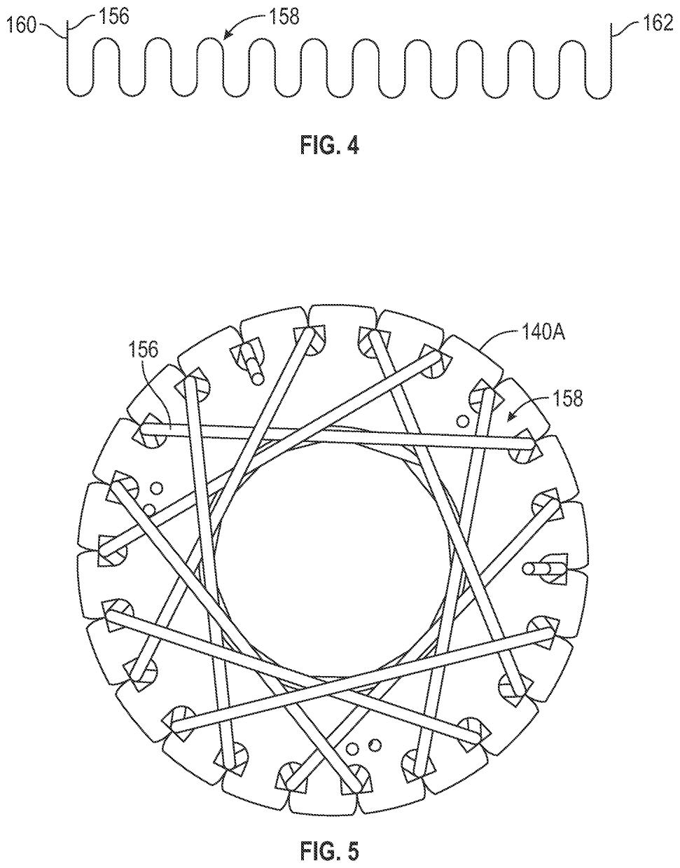

Referring also to FIGS. 3A-3D, the gas extraction assembly 102 includes one or more retainers 140A and 140B, collectively referred to herein as retainers 140. In the present embodiment, two retainers 140A and 140B are illustrated positioned within the interior space 122 of the extraction chamber 116, however, in other embodiments, more or fewer retainers 140 may be used. The retainers 140A and 140B may be generally flat, round discs or O-rings and may be constructed of polymeric material, such as VITON rubber, while other materials may be used in other embodiments. The retainers 140A and 140B have an outer circumference edge 142 and an inner circumference edge 144. The inner circumference edge 144 defining an opening 146 in the retainers 140A and 140B. The retainers 140A and 140B include an upper surface 148, a lower surface 150, and a plurality of guides 152 or openings that extend through from the upper surface 148 through the lower surface 150. The plurality of guides 152 are located adjacent the outer circumference edge 142 in this embodiment, but may be located elsewhere in other embodiments. A plurality of slits 154 or cuts are provided in the retainer 140 adjacent each of the guide 152 openings. Each of the slits 154 cut through the retainer 140 and extend linearly from the each of the guides 152 to the outer circumference edge 142. As will be discussed, the slits 154 provide for ease of assembly, but may be omitted in other embodiments.

The retainers 140A and 140B are positioned in the extraction chamber 116 such that the inner circumference edge 144 of the retainers 140A and 140B are positioned adjacent and abuts the outer wall 131 of the supply conduit 126, and further such that the outer circumference edge 142 of the retainers 140A and 140B are positioned adjacent and abuts the inner wall 133 of the extraction chamber 116. Referring to FIG. 3D, the supply conduit 126 is shown with the retainers removed to illustrate outer grooves 155A and 155B configured circumferentially about the outer wall 131 of the supply conduit 126. The retainers 140A and 140B are sized to be positioned in the outer grooves 155A and 155B, respectively, of the supply conduit 126 to be retained in position on the supply conduit 126. In some embodiments, it may be useful to maintain a minimal gap between the retainers 140 and the outer wall 131 of the supply conduit 126 to prevent movement, such as twisting, so the supply conduit 126 does not tug or pull and potentially damage the gas permeable membrane 156. While two retainers 140A and 140B are shown, fewer or more retainers 140 constructed of similar or other materials, may be use and positioned anywhere between the upper end 118 and lower end 120 of the extraction chamber 116.

A gas permeable membrane 156 is shown disposed in S-shaped curves extending between adjacent the upper and lower ends 118 and 120 of the extraction chamber 116. The gas permeable membrane 156, which may be any gas permeable material, may be a desired length of tubular material that allows dissolved gases in the fluid 112 to permeate into the interior passageway of the tubular gas permeable membrane 156 while preventing the ingress of liquids in the fluid 112. The gas permeable membrane 156 may be a TEFLON AF (amorphous perfluoropolymer) membrane or other suitable gas permeable material.

Referring also to FIGS. 1 and 2, the gas permeable membrane 156 is retained in position within the extraction chamber 116 via retainers 140A and 140B. In this embodiment, the gas permeable membrane 156 extends through the guides 152 in the retainers 140A and 140B in a series of S-shaped curves. For example, the gas permeable membrane 156 may extend down through one of the guides 152 in the retainer 140A and continue down through one of the guides 152 in the retainer 140B that is positioned directly below the guides 152 in the retainer 140A. Thereafter, the gas permeable membrane 156 curves and returns, extending up, through another, different one of the guides 152 in the retainer 140B and up through another one of the guides 152 in the retainer 140A that is positioned directly above the guides 152 in the retainer 140B, and so on until the gas permeable membrane 156 has been positioned through all the guides 152 in the retainers 140A and 140B.

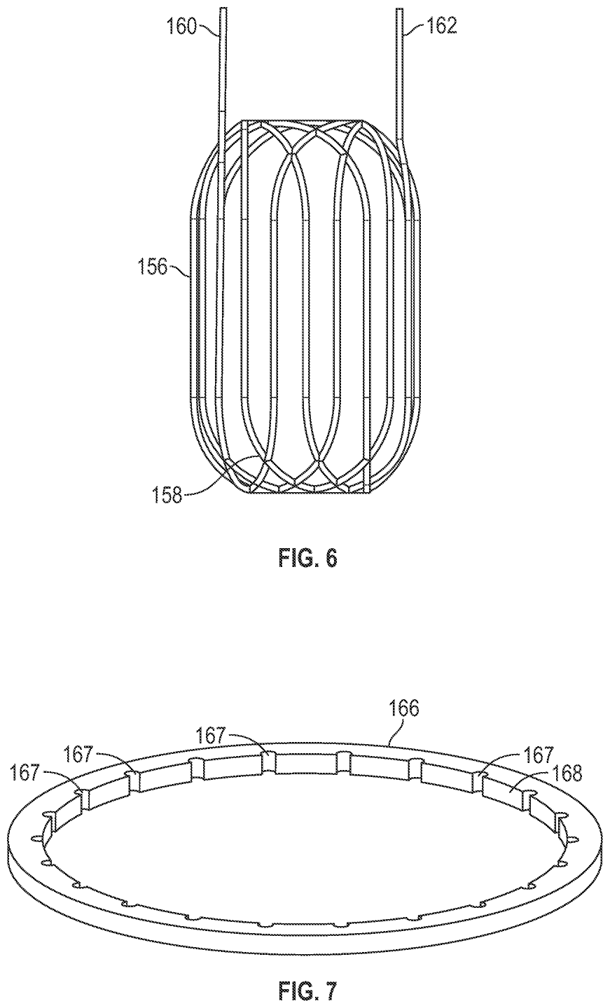

FIG. 4 depicts the gas permeable membrane 156 laid-out flat to illustrate the S-shaped curves. The gas permeable membrane 156 includes a first end 160 and a second end 162. FIG. 5 illustrates a top view of the gas permeable membrane 156 positioned in the S-shaped curves 158 and held by the retainers 140A and 140B. FIG. 6 is a side perspective view of the gas permeable membrane 156 illustrated in the S-shaped curves 158 with the retainers 140A and 140B removed in this view. As can be seen, the S-shaped curves 158 do not intersect one another but instead the gas permeable membrane 156 inner or outer layers merely overlap one another. In some embodiments, the gas permeable membrane 156 may overlap multiple times such as, seven (7) times, while in other embodiments more or fewer overlaps may be used depending on the overall size of the gas extraction assembly 102, extraction chamber 116, and desired length of the gas permeable membrane 156 to be used. Where more or fewer loops or layers are desired, the retainers 140 would be appropriately configured.

It will be appreciated that the gas permeable membrane 156 may be flexible and bend under tension into the S-shaped curves, but when not constrained gas permeable membrane 156 may elastically return to an original or natural shape, such as generally straight. Thus, it may be challenging for a user to assemble the gas permeable membrane 156 in the retainers 140 to achieve the multiple S-shaped curves 158 to form the overall configuration, which may be referred to herein as a “bird-cage” type overall configuration, as shown in FIGS. 1-2 and 5-6. Accordingly, referring back to FIG. 3, the slits 154 in the retainers 140 may assist the user in positioning the gas permeable membrane 156 into the guides 152 in the retainers 140. For example, the user may slide or press a portion of the gas permeable membrane 156 into one of the slits 154 and further into the adjacent guide 152 in order to position that portion of the gas permeable membrane 156 into the guide 152.

During assembly, the gas permeable membrane 156 may be threaded or positioned into guides 152 generally across from one another instead of next to one another, in order to prevent crimping the gas permeable membrane 156 and potentially restricting the flow of gases traveling through the gas permeable membrane 156. To assist the user in consistently threading the gas permeable membrane 156 through the retainers 140, indicia 164A-164C may be provided on the upper and/or lower surfaces 148, 150 of the retainers 140. For example, the user may begin threading the gas permeable membrane 156 upward, for example, through the guides 152 adjacent the indicia 164A of the retainers 140A and 140B, then loop across and down through the guides 152 adjacent the indicia 164B, and then loop across and up through the guides 152 adjacent indicia 164C. Thereafter, the user may simply rotate or index to the next or neighboring guide 152 adjacent the guide 152 nearest the previous indicia 164A-C and repeat the steps of providing the gas permeable membrane 156 in S-shaped curves until the gas permeable membrane 156 has been threaded through all the guides 152 in the retainers 140A and 140B to form the overall bird cage configuration. In this embodiment, about 2 meters of gas permeable membrane 156 may be used, however more or less may be used in other embodiments.

FIG. 7 illustrates a support member 166, which is also shown in FIGS. 1-3. The support member 166 is a substantially flat O-shaped ring constructed of rubber in this embodiment, but other materials could be used. As can be seen in FIGS. 3B and 3C, the support members 166 may be placed adjacent above and/or below each of the retainers 140A and 140B. In this embodiment, the support members 166 include support groves 167 provided on an inner surface 168 of the support member 166. The number and location of the support groves 167 are configured to correspondingly align with the guides 152 in the retainers 140. The support members 166 are positioned between the gas permeable membrane 156 extending through the guides 152 in the retainers 140 and the inner wall 133 of the extraction chamber 116. In this position, the support member 166 acts to constrain the elasticity of the gas permeable membrane 156 from expanding into contact with the inner wall 133 of the extraction chamber 116 and retains the gas permeable membrane 156 positioned in the retainers 140. It will be appreciated that the gas permeable membrane 156 may be fragile and this design may prevent friction or rubbing of the gas permeable membrane 156 with surfaces within the extraction chamber 116. In addition, the support member 166 further acts to retain the gas permeable membrane 156 positioned in the retainers 140 and prevents the gas permeable membrane 156 from elastically slipping out of or exiting the guides 152 via slits 154 in the retainers 140. It will be appreciated that general “bird cage” configuration of the present disclosure allows for exposure of more surface area of the gas permeable membrane 156 to the fluid 112 which aids in promoting improved extraction of gas into the gas permeable membrane 156.

Returning to FIGS. 3A-3C, the overall size of the of the guides 152 openings may be a matter of design choice. The openings defining the guides 152 are, at a minimum, sized such that the gas permeable membrane 156 may pass through the guide 152 openings. However, to promote improved extraction of gas into the gas permeable membrane 156, the guide 152 openings may be larger, for example about two (2) to three (3) times or more larger, than the diameter of the gas permeable membrane 156. As can be seen in FIGS. 3B-3C, this additional space provides for sufficient flow of the fluid 112 between each guide 152 opening and the gas permeable membrane 156 while keeping the flow of fluid 112 sufficiently close to the gas permeable membrane 156 to promote gas extraction. This configuration and design of the gas permeable membrane 156 positioned, via the retainers 140 and support member 166, in the extraction chamber 116 may further improve, or in some embodiments maximize, the surface area of the gas permeable membrane 156 that is exposed to the fluid 112 as it is flowed through the extraction chamber 116 which promotes improved extraction of the gas in the fluid 112. FIGS. 3B-3C further illustrates the separation or spreading of the gas permeable membrane 156 when so positioned in the “bird cage” configuration. Thus, while the gas permeable membrane 156 may occasionally overlap and contact along S-shaped curves, the gas permeable membrane 156 is primarily separated from itself or “floats in space” so as to maximize the surface area of the gas permeable membrane 156 exposed to the fluid 112, which promotes improved gas extraction. For example, in some embodiments, a flow of the fluid 112 through the gas extraction chamber 116 of a gallon per minute, less than a gallon per minute, or perhaps one-tenth ( 1/10) of a gallon per minute or less, may be sufficient to obtain the desired extraction of gas, while other flow rates may be sufficient in other embodiments.

Referring again to FIGS. 1 and 2, the gas permeable membrane 156 is coupled at first and second ends 160 and 162 to communicate the diffused gas extracted from the fluid 112 to the gas analyzer 103 via first and second lines 170 and 172. First and second lines 170 and 172 may be metal such as stainless-steel piping or tubing. The first and second ends 160, 162 of the gas permeable membrane 156 and first and second lines 170, 172 may be connected via fittings 174 attached to a lower side 176 and the upper side of the cap 124. The fittings 174 may be any fittings or adapters to couple to the first and second ends 160, 162 of the gas permeable membrane 156 and first and second lines 170, 172 to communicate the diffused gas therethrough. The cap 124 may include channels or passageways between the fittings 174 to allow for communication of diffused gasses between respective connections on the lower side 176 and upper side 125 of the cap 124.

The gas analysis system 100 may, in some embodiments, include a pump 178. The pump 178 is connected to the manifold 104 and configured to promoted the flow of the fluid 112 through the gas analysis system 100. The pump 178 may be any type of pump capable of pumping the fluid 112, such as diaphragm, centrifugal, or gear pump. As shown in FIG. 2, the manifold 104 includes an inlet channel 180 that extends from a manifold inlet 181 on an angled interface 182 of the manifold 104 to a filter channel 184 in the manifold 104. The angled interface 182, which will be described in more detail herein, couples the manifold 104 to receive the fluid 112 from the transformer 106. The inlet channel 180 and filter channel 184 are fluid tight passageways within the manifold 104.

The filter channel 184 extends from a lower side 188 to an upper side 190 of the manifold 104. A filter 186 is provided in the filter channel 184 near the lower side 188 of the manifold. The filter 186 may be a filter or strainer configured to remove particulates from the fluid 112 before entering the pump 178. A filter plug 187 is shown connected, such as via threaded attachment, to fluidly seal the filter channel 184 adjacent the lower side 188 of the manifold 104. The filter plug 187 may be removed to allow access to the filter 186 for replacement or cleaning.

The pump 178 is attached adjacent the upper side 190 of the manifold 104 at a pump inlet 192. Fluid 112 drawn from the transformer 106, via the pump 178, exits the pump 178 at a pump outlet 194. The pump outlet 194 is in fluid communication with a transverse channel 196, via a pump line 198 connected therebetween via couplings 200, 202. The pump line 198 may be metal such as stainless-steel piping or tubing. The couplings 200, 202 may be any fittings or adapters to couple to the pump outlet 194 and the transvers channel 196 to communicate fluid 112 therethrough. Transverse channel 196 is a fluid tight passageway in the manifold 104 that extends from adjacent coupling 202 of pump line 198 to adjacent the supply conduit 126 to the manifold 104. More specifically, the transverse channel 196 is in fluid communication with the passageway 138 at a lower end the supply conduit 126. Thus, the fluid 112 from the transformer 106 is communicated through the manifold 104 and pump 178 into the passageway 138 of the supply conduit 126 and exits through holes 136 of the supply conduit 126 into the extraction chamber 116. Fluid exiting through holes 136 near upper end 118 of the extraction chamber 116 flows downward toward the lower end 120 of the extraction chamber 116 and reenters the manifold 104 via outlet channel 206. The outlet channel 206 extends from the lower end 120 of the extraction chamber 116 to a manifold outlet 207 at the angled interface 182 of the manifold 104 where it is returned to the transformer 106, as well be further discussed below.

It will be appreciated that the location of the pump 178 and the various fluid communication paths of the manifold 104 may differ in other embodiments and thus, the present embodiment is not limited to the particular configuration described above. For example, in some embodiments, the pump 178 may be omitted, and instead the fluid 112 from the transformer 106 may be communicated through the gas analysis system 100 due to buoyant forces created by temperature gradients in the fluid 112. In such an embodiment, for example, the filter channel 184, and the intervening fluid passageway and components may be omitted. These and other embodiments are contemplated and will readily suggest themselves to one skilled in the art in view of the present teachings.

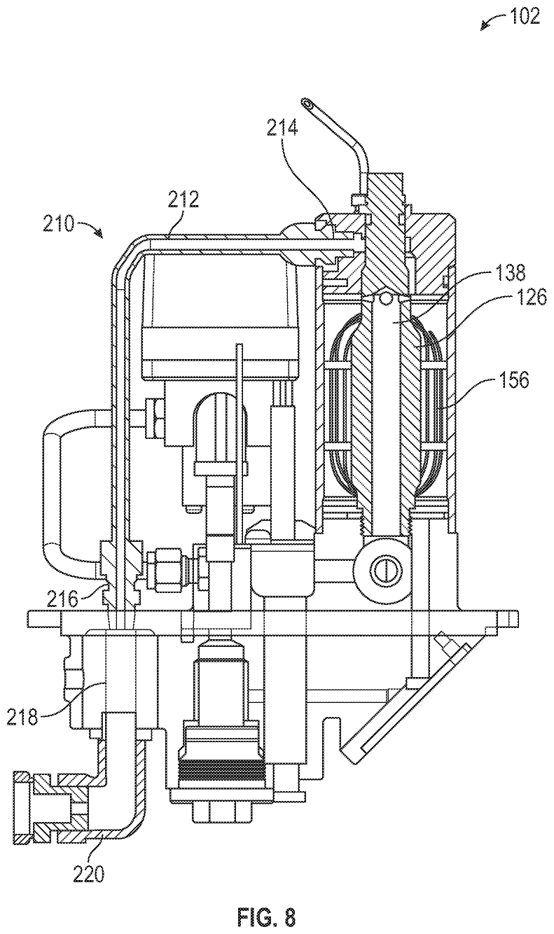

FIG. 8 is a view of the back side of the gas analysis system 100 of the present disclosure. In this embodiment and the embodiment shown in FIG. 1, the gas analysis system 100 includes a vent system 210. The vent system 210 includes a vent line 212 connected and in communication adjacent a first end 214 with the supply conduit 126. More specifically, the vent line 212 is coupled and in fluid communication with the passageway 138 within the supply conduit 126. The location of the connection of the vent line 212 to the supply conduit 126, may in this embodiment, represent the highest point of the fluid 112 flow path through the gas analysis system 100. In this embodiment, the vent line 212 is connected at a second end 216 to the upper side 190 of the manifold 104. The vent line 212 communicates at the second end 216 with a vent channel 218 that extends within the manifold 104 from the upper side 190 to the lower side 188 of the manifold 104. A sampling port 220 may be connected, such as via a threaded connection, to the lower side 188 of the manifold 104 and be in fluid communication with the vent channel 218. The sampling port 220 may be variously configured, such as via elbows, unions, caps, and ports, such as a sampling and/or air purge port 222. Sampling and/or air purge port 222 is openable to the surrounding area.

It will be appreciated that when the gas analysis system 100 is initially connected to the transformer 106, serviced, or otherwise, the manifold 104 and gas extraction assembly 102 may contain air that may need to be purged out of the gas analysis system 100 to prevent air from entering the transformer 106 or for other reasons. It can be seen that, by opening the sampling and/or air purge port 222, the vent system 210 allows the static head pressure of the fluid 112 to flow into the gas analysis system 100 which causes air to be expelled from the gas analysis system 100. The pump 178 may be optionally operated at low speed to assist during this process. Further, periodically transformers 106 may require that samples of the fluid 112 or oil in the transformer 106 be taken for analysis. The fluid 112 samples may be retrieved via the sampling and/or air purge port 222.

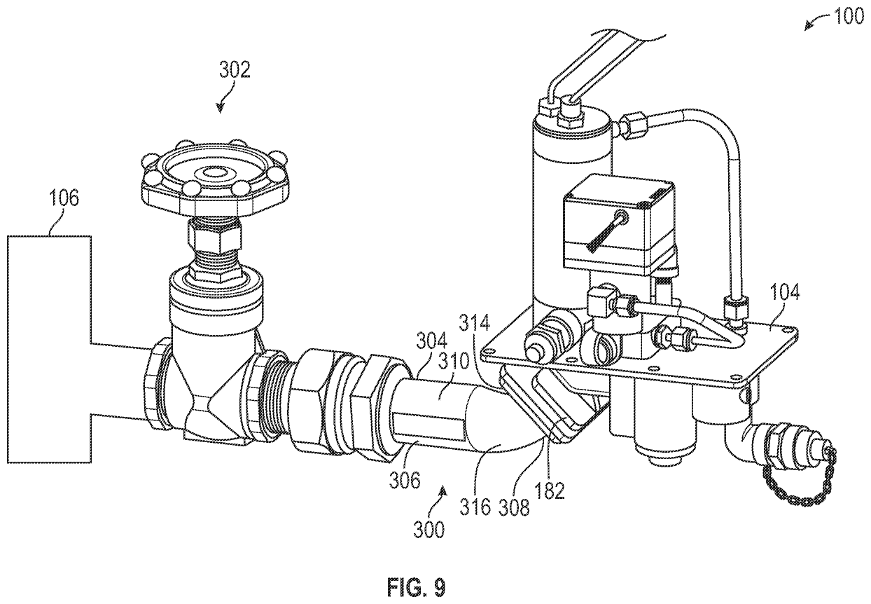

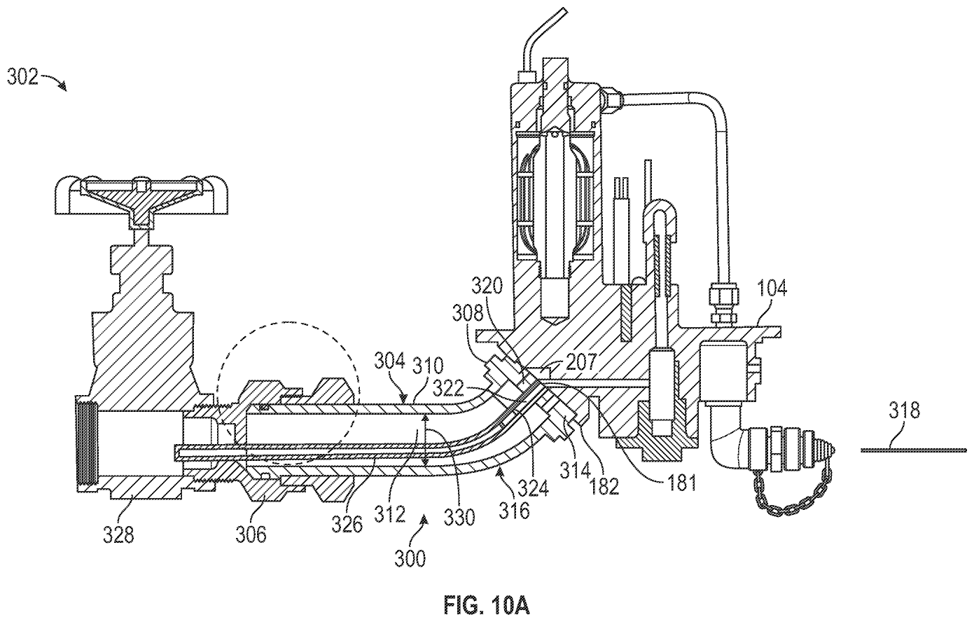

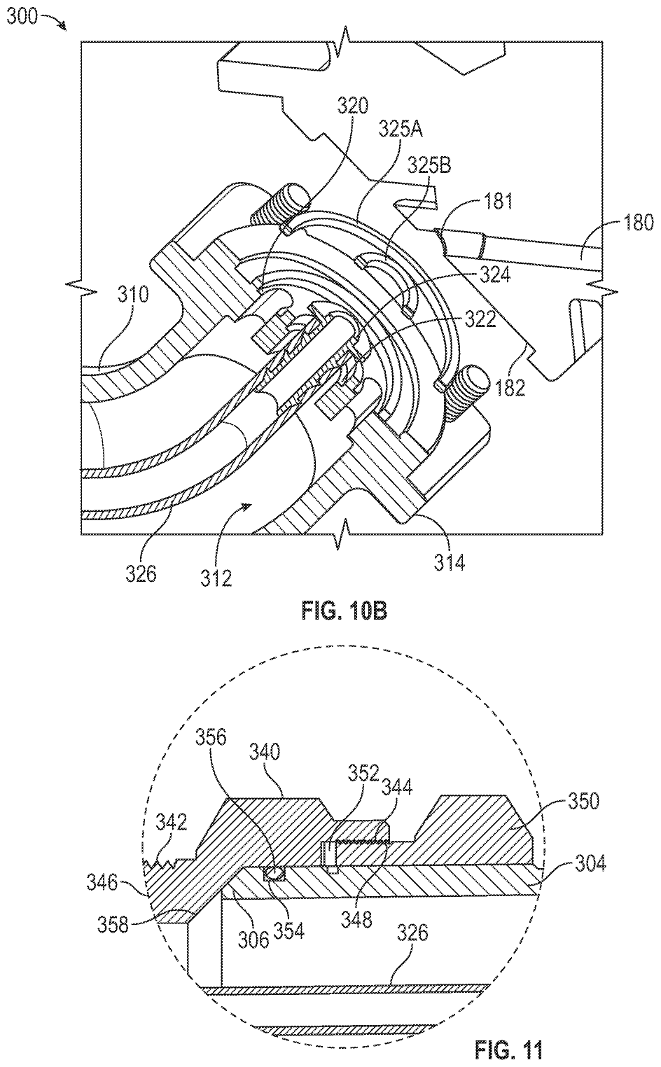

While some transformers 106 may include ports, such as valve 114, specifically provided on the transformer 106 for connecting systems, such as the gas analysis system 100, other transformers 106 may only include drain or other valves or connection points. FIGS. 9 and 10A-10B illustrate an attachment system 300 for connecting the gas analysis system 100 to a drain valve 302 of the transformer 106. Although the drain valve 302 is illustrated as a gate or ball valve, other well-known ports or valves may be used in other embodiments. The drain valve 302 is connected to and in fluid communication with the fluid 112 within the transformer 106.

In the present embodiment, the gas extraction assembly 102 is oriented or extends generally vertically relative to manifold 104 and a horizontal plane 318. Such orientation of the gas analysis system 100 may be referred to as a “vertical” orientation. The present disclosure is not limited to this orientation and other positioning may be used in other embodiments. In the “vertical” orientation shown in the present embodiment, it can be seen that the angled interface 182 of the manifold 104 is angled relative to the horizontal plane 318 at an angle, such as 45 degrees (45°). Thus, the angled interface 182 may be said to be at an incline or acute angle relative to the horizontal plane 318.

The attachment system 300 includes an adapter fitting 304 connected at a first end 306 adjacent the drain valve 302 and connected at a second end 308 to the angled interface 182 of the manifold 104 of the gas analysis system 100. The adapter fitting 304 may be a tubular pipe or fitting having an outer surface 310 defining a fluid passageway 312 extending between the first and second ends 306 and 308 of the adapter fitting 304. An interface 314 is provided at the second end 308 of the adapter fitting 304 to mate with the angled interface 182 of the manifold 104. The adapter fitting 304 extends as an elongate section from the first end 306 toward a bend portion 316 adjacent the second end 308 at a bend of approximately 45 degrees (45°). The bend portion 316 allows the adapter fitting 304 to extend from the first end 306 horizontally, relative to the horizontal plane 318, straight from the drain valve 302 and angles at the bend portion 316 so that the interface 314 at the second end 308 angularly mates with the angled interface 182 of the manifold 104. The interface 314 of the adapter fitting 304 is attachable to angled interface 182 of the manifold 104 via screws, bolts, threaded or other well-known systems for accomplishing a mechanical connection and fluid tight connectors such as O-rings for a liquid tight connection.

As can be seen in FIGS. 10A-10B, the interface 314 includes an interface inlet 320 and an interface outlet 322. When the interface 314 is connected to the angled interface 182 of the manifold 104, the interface inlet 320 is in fluid communication with the manifold outlet 207 and the interface outlet 322 is in fluid communication with the manifold inlet 181. The interface outlet 322 may include a hose nipple 324 captured between the interface 314 of the adapter fitting 304 and angled interface 182 of the manifold 104 and extending from the interface outlet 322 into the fluid passageway 312. The interface 314 of the adapter fitting 304 may include various rubber gaskets or O-rings 325A and 325B on interface inlet and outlet 320 and 322 to promote a fluid tight seal. An extension tube 326 or pipe may be connected to the hose nipple 324 and extend through the fluid passageway 312 to adjacent the first end 306 of the adapter fitting 304. The extension tube 326 allows fluid to flow within the extension tube 326 from the hose nipple 324 to adjacent the first end 306 of the adapter fitting 304. As can be seen, the extension tube 326 has a smaller outer diameter 328 than the larger inner diameter 330 of the adapter fitting 304. In this embodiment, the extension tube 326 may be a flexible rubber (e.g. Viton®) or plastic (e.g. polytetrafluoroethylene) tube or hose, but may be rigid, for example metal, such as stainless steel, brass, carbon steel, in other embodiments.

FIG. 11 is an expanded cut-away portion shown in FIG. 10A taken along “C” illustrating one embodiment of the coupling of the attachment system 300 to the drain valve 302. In this embodiment, a union receiver 340 is provided that includes a first threaded portion 342 and a second threaded portion 344. The first threaded portion 342 is connected to drain valve 302 adjacent a first end 346 of the union receiver 340. The first end 306 of adapter fitting 304 is positioned within the union receiver 340. The second threaded portion 344 of the union receiver 340 is connected to a threaded portion 348 of a union nut 350 that is provided to surround the adapter fitting 304 adjacent the first end 306. A retaining ring 352 is located adjacent the threaded interface of the union receiver 340 and union nut 350 to transfer thrust from the union nut 350 to the adapter fitting 304 driving the adapter fitting 304 into engagement with the union receiver 340.

As can be seen, the union receiver 340 includes a shoulder 358 that provides a primary fluid sealing point of the union receiver 340 with the first end 306 of the adapter fitting 304. The shoulder 358 also provides a ramp or slope that allows the extension tube 326 to extend beyond the interface of the union receiver 340 and first end 306 of the adapter fitting 304 without getting caught or crimped at that point. In this embodiment, the adapter fitting 304 may include a notch 354 adjacent the first end 306 of the adapter fitting 304 for receiving an O-ring 356, such as a rubber gasket O-ring, to provide for further fluid sealing of the connection.

As can be seen, the attachment system 300 is configured such that the first end 306 generally terminates vertically, relative to the horizontal plane 318, while the second end 308, at the attachment interface 314, generally terminates at an angle, such as 45 degrees (45°) relative to the horizontal plane 318, to angularly mate with the angled interface 182 of the manifold 104.

In practice, when the drain valve 302 is opened, fluid 112 from the transformer 106 is drawn into the extension tube 326 adjacent the first end 306 of the adapter fitting 304. The fluid 112 traverses the extension tube 326, via static pressure, the pump 178, or otherwise, exits the attachment system 300, via the hose nipple 324 and the interface outlet 322, and enters the gas analysis system 100, via manifold inlet 181. After analysis by the gas analysis system 100, the fluid 112 exits the gas analysis system 100, via manifold outlet 207, and enters the attachment system 300, via interface inlet 320, and flows into the fluid passageway 312 within the adapter fitting 304.

Thus, according to the present disclosure, the fluid 112 is drawn into the extension tube 326 which is located closer to the transformer 106 than the interface 314 adjacent the second end 308 of the adapter fitting 304. It will be appreciated that there may be advantages, in some embodiments, for having the gas analysis system 100 and the fluid 112 being sampled, in close physical proximity to the transformer 106 so that the fluid 112 from the transformer 106 being analyzed is most representative of the fluid 112 in the transformer 106. Further, once the fluid 112 has been tested by the gas analysis system 100, it is returned to the fluid passageway 312 adjacent the second end 308 of the adapter fitting 304. Since the gas analysis system 100 removes certain amounts of the gas in the fluid 112 for testing, the returned fluid 112 is no longer representative of the gas content of the fluid 112 as it exists in the transformer 106. By drawing the fluid 112 to be sampled or tested at a point closer to the transformer 106, a more representative sample of fluid and consequently a more accurate test may be conducted in contrast to drawing the fluid 112 to be sampled from the same location as the returned, gas extracted fluid 112. Furthermore, as the returned or tested fluid 112 enters the fluid passageway 312, the fluid 112 remixes with the fluid 112 from the transformer 106 forced into the fluid passageway 312. Thus, even after time, the fluid 112 drawn into the extension tube 326 continues to be representative of the fluid 112 in the transformer 106, which promotes more accurate ongoing testing of the fluid 112.

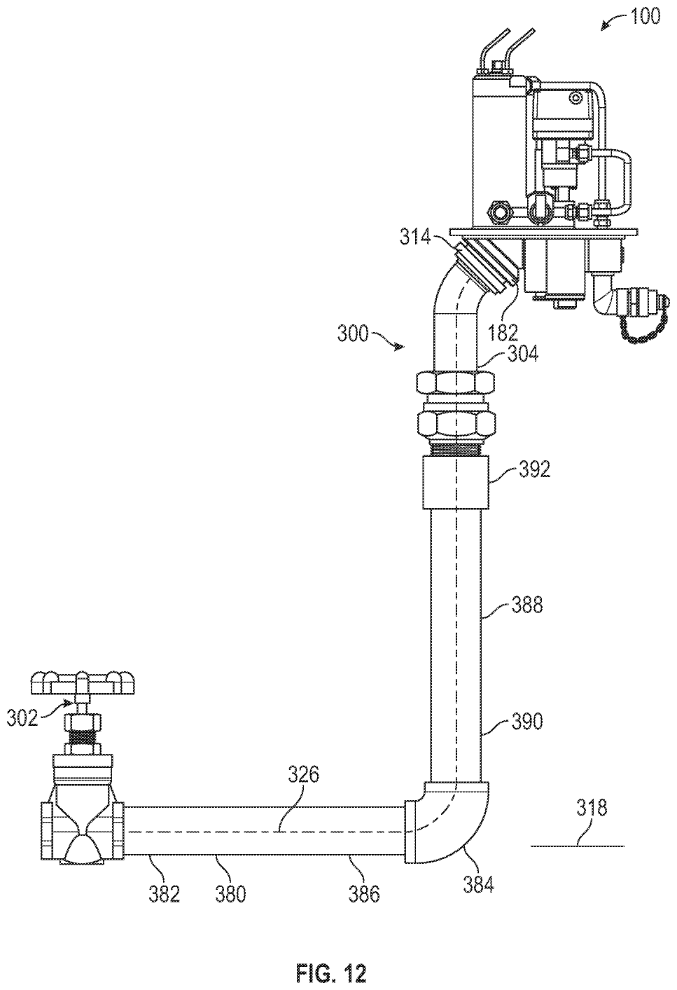

While the gas analysis system 100 is generally shown in FIGS. 9 and 10 positioned horizontally, relative to the horizontal plane 318, extending from the drain valve 302 of the transformer 106, in some instances it may be useful to provide the gas analysis system 100 at other locations due to the space available in surrounding areas or for other reasons. FIG. 12 illustrates another implementation of the present disclosure. In this embodiment, a first pipe 380 is shown attached at a first end 382 to the drain valve 302. The first pipe 380 extends from the drain valve 302 horizontally, relative to the horizontal plane 318, and connects to an elbow 384 at a second end 386 of the first pipe 380. A second pipe 388 is attached at a first end 390 of the second pipe 388 to the elbow 384 and at a second end 392 of the second pipe 388 to the attachment system 300. The connection between attachment system 300 and the second end 392 of the second pipe 388 is accomplished similar to that described in FIGS. 9-11 with regard to the connection of the attachment system 300 and the drain valve 302.

As can be seen in FIG. 12, the attachment system 300 is oriented generally vertically, relative to the horizontal plane 318, instead of generally horizontally, as shown in FIGS. 9 and 10. Due to the angular configuration of the angled interface 182 of the manifold 104 and angled interface 314, the adapter fitting 304 of the attachment system 300 may simply be re-oriented to reposition the gas analysis system 100 in such position or location while still maintaining the “vertical” orientation of the gas analysis system 100.

In this embodiment, the extension tube 326 may extend from adjacent the manifold 104 through the attachment system 300, second pipe 388, elbow 384, first pipe 380, and terminate adjacent the drain valve 302. In this manner, the fluid 112 is drawn into the extension tube 326 which is located closer to the transformer 106 than the interface 314 adjacent the second end 308 of the adapter fitting 304. As mentioned above, this provides for the fluid 112 being sampled to be in close physical proximity to the transformer 106 so that the fluid 112 from the transformer 106 being analyzed is most representative of the fluid 112 in the transformer 106. Thus, even though the gas analysis system 100 is located farther away or remote from the transformer 106, the present system continues to provide for sampling of the fluid 112 nearer the transformer 106, which may provide advantages as discussed above. It will be appreciated that the present disclosure is not so limited and, in other embodiments, the extension tube 326 may extend more or less far, such as only into the second pipe 388 for example.

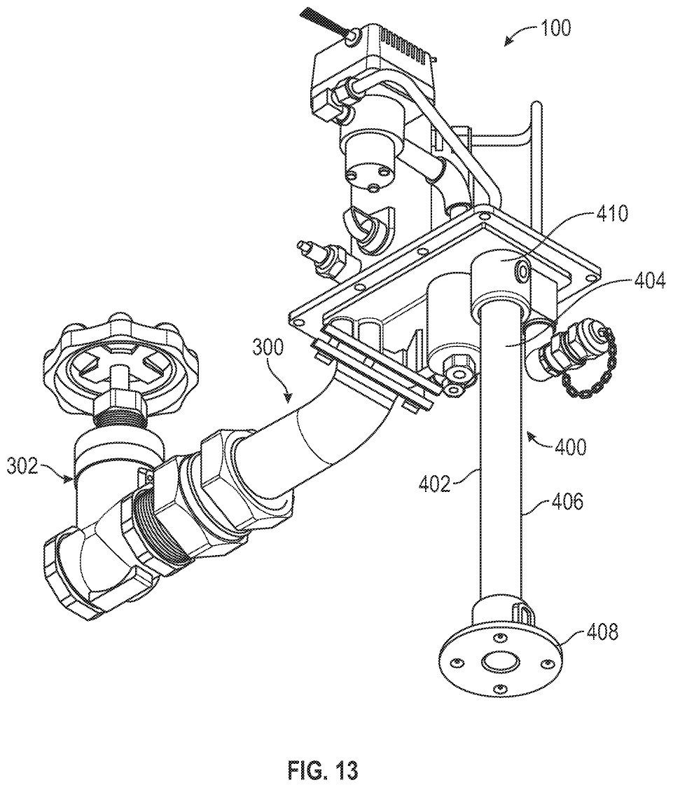

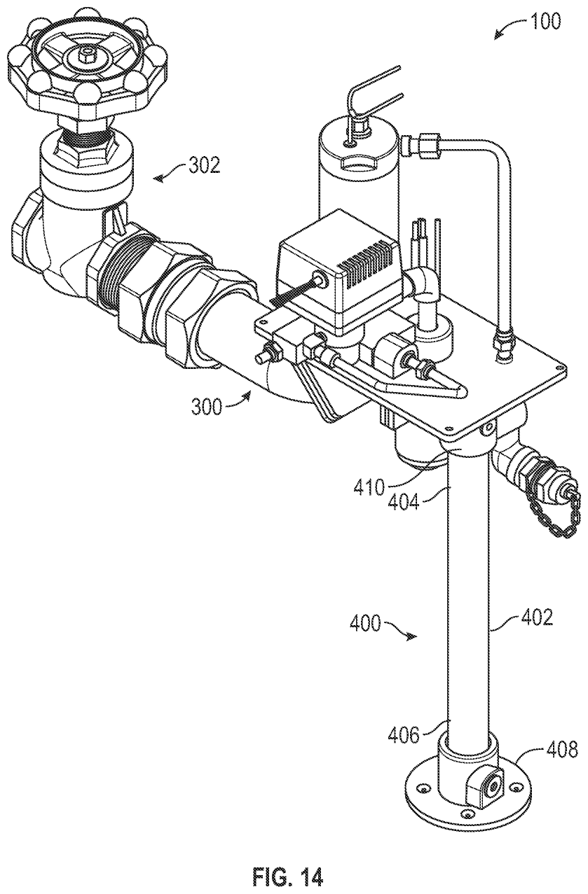

Referring to FIGS. 13 and 14 another embodiment of the present disclosure is shown. It will be appreciated that the weight of the gas analysis system 100 and attachment system 300 may place stress on the drain valve 302, such as small size valves, or other attachment points of the transformer 106. As shown in FIGS. 13 and 14, the present disclosure provides a support system 400 to support the weight of the gas analysis system 100 and attachment system 300. The support system 400 may include a support arm 402, which may be a pipe or other piece, attached at an upper end 404 to the lower side 188 of the manifold 104 and at a lower end 406 to a floor fitting 408 of the support system 400.

In this embodiment, the manifold 104 includes a socket 410 which may be integrally formed or cast into the manifold 104 and configured to receive the upper end 404 of the support arm 402. The support arm 402 may be secured to the socket 410 of the manifold 104 at the upper end 404 and the floor fitting 408 at the lower end 406 via tensioning screws or otherwise. The floor fitting 408, such as a floor flange or rail fitting, is configured for attachment or engagement with a support surface, such as the ground or concrete pad, adjacent the transformer 106. In this manner, the weight or load of the gas analysis system 100 and attachment system 300 is transferred to the support surface relieving pressure or mechanical strain on the drain valve 302 and the attachment system 300 connection or interface 314.

While the embodiments shown in FIGS. 13 and 14 show the attachment system 300 and gas analysis system 100 in the generally horizontal position, the support system 400 may be readily adapted, such as by providing a longer support arm 402 for supporting the gas analysis system 100 when provided in the vertical position as shown in FIG. 12. Depending on the desired application, it will be appreciated that additional pipe or coupling pieces or other makeup fittings may be used in various configurations in other embodiments as will readily suggest themselves to one skilled in the art, all of which are within the spirit and scope of the present disclosure.

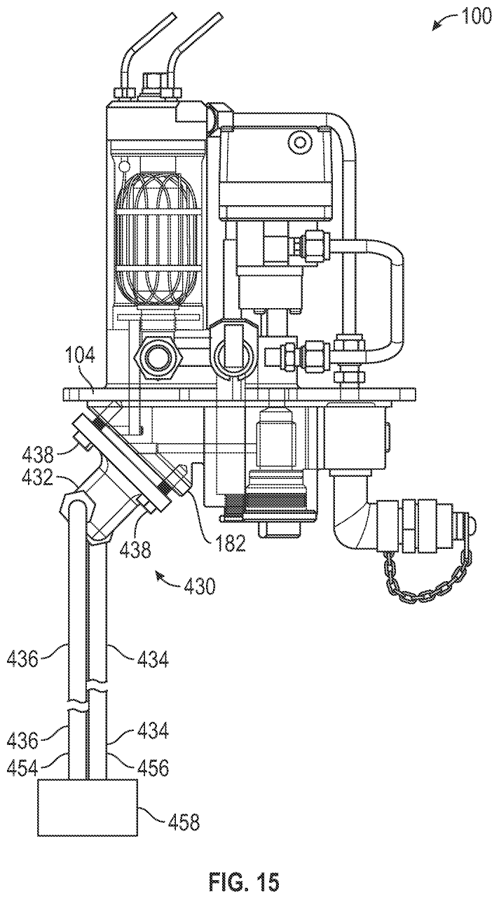

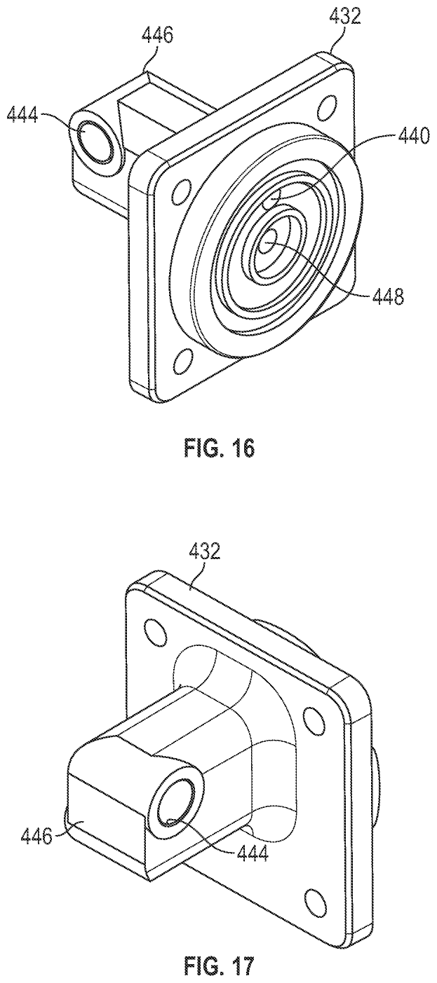

The present disclosure further contemplates deployment of the gas analysis system 100 in locations more remote than is practicable using only the attachment system 300 and pipes and fittings shown in FIGS. 9, 10, and 12. Instead, as shown in FIG. 15, a remote attachment system 430 is provided. The remote attachment system 430 includes a mount adapter 432, a supply line 434, and a return line 436. The mount adapter 432 is shown attached to the angled interface 182 of the manifold 104 of the gas analysis system 100. The mount adapter 432 is coupled to the angled interface 182 via screws 438, but may be otherwise attached in other embodiments.

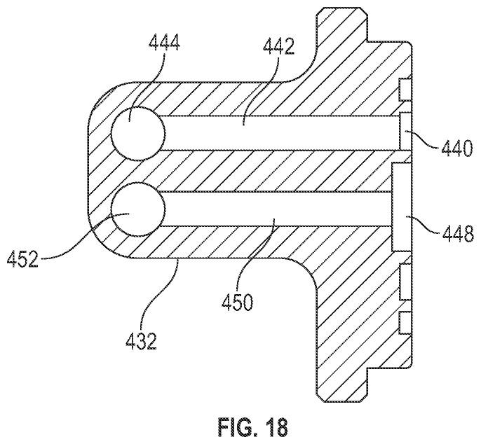

FIGS. 16-18 illustrate various views of the mount adapter 432. The mount adapter 432 includes a return port 440 that is in fluid communication with the manifold outlet 207 when the mount adapter 432 is coupled to the angled interface 182. A return channel 442 extends within the mount adapter 432 between the return port 440 and a return outlet 444. The return outlet 444 is provided adjacent an adapter rear side 446 substantially opposite the return port 440. The return line 436 fluidly couples to the return outlet 444.

The mount adapter 432 also includes a supply port 448 that is in fluid communication with the manifold inlet 181 when the mount adapter 432 is coupled to the angled interface 182. A supply channel 450 extends within the mount adapter 432 between the supply port 448 and a supply outlet 452. The supply outlet 452 is provided adjacent the adapter rear side 446 substantially opposite the supply port 448. The supply line 434 fluidly couples to the supply outlet 452.

First ends 454, 456 of the return and supply lines 436, 434, respectively, may fluidly couple to a remote interface 458. The remote interface 458 may be configured to couple directly to the drain valve 302 or other connection points of the transformer 106 or via the attachment system 300. For example, the first ends 454, 456 of the return and supply lines 436, 434, respectively, may fluidly couple to a single drain valve 302 or separate oil inlet and oil outlets of the transformer 110.

The remote attachment system 430, like the various other systems and components described herein, such as the attachment system 300 and gas analysis system 100, may be constructed of brass, steel, stainless steel, polymeric, rubber, or other well-known materials as desired or as described herein.

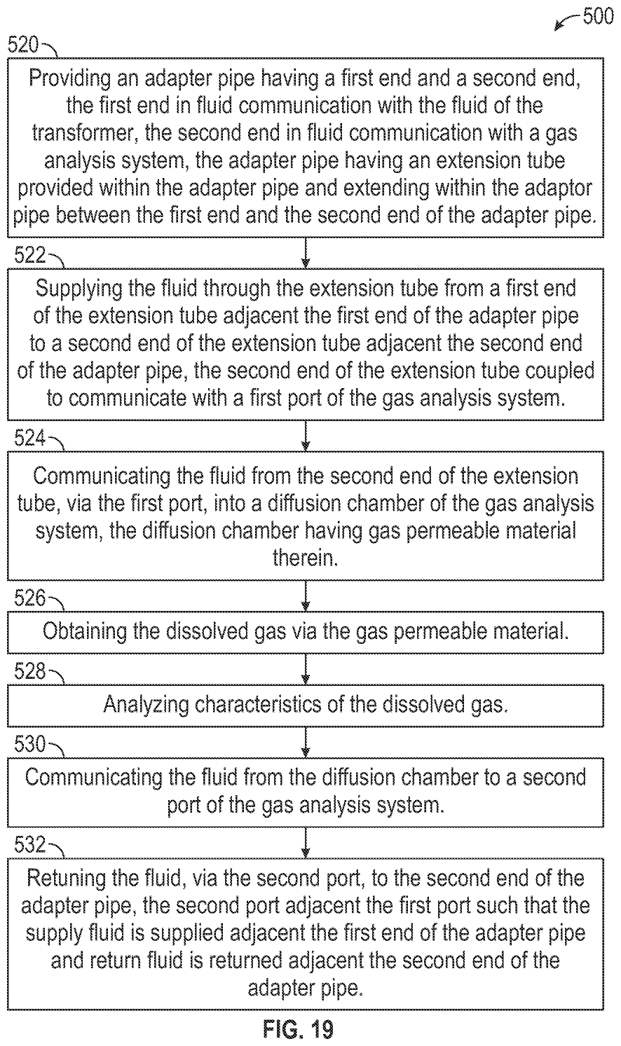

Referring to FIG. 19, a method 500 for analyzing dissolved gases in a fluid of a transformer is shown. The method includes, at box 520, providing an adapter pipe having a first end and a second end, the first end in fluid communication with the fluid of the transformer, the second end in fluid communication with a gas analysis system, the adapter pipe having an extension tube provided within the adapter pipe and extending within the adapter pipe between the first end and the second end of the adapter pipe. The method includes, at box 522, supplying the fluid through the extension tube from a first end of the extension tube adjacent the first end of the adapter pipe to a second end of the extension tube adjacent the second end of the adapter pipe, the second end of the extension tube coupled to communicate with a first port of the gas analysis system. The method includes, at box 524, communicating the fluid from the second end of the extension tube, via the first port, into an extraction chamber of the gas analysis system, the extraction chamber having gas permeable material disposed therein. The method includes, at box 526, obtaining the dissolved gas via the gas permeable material. The method includes, at box 528, analyzing characteristics of the dissolved gas. The method includes, at box 530, communicating the fluid from the extraction chamber to a second port of the gas analysis system. The method includes, at box 532, returning the fluid, via the second port, to the second end of the adapter pipe, the second port adjacent the first port such that the supply fluid is supplied adjacent the first end of the adapter pipe and return fluid is returned adjacent the second end of the adapter pipe. In some embodiments, the fluid may be returned to fitting not adjacent the first end, such as higher or lower on the transformer or elsewhere for communication to the transformer.

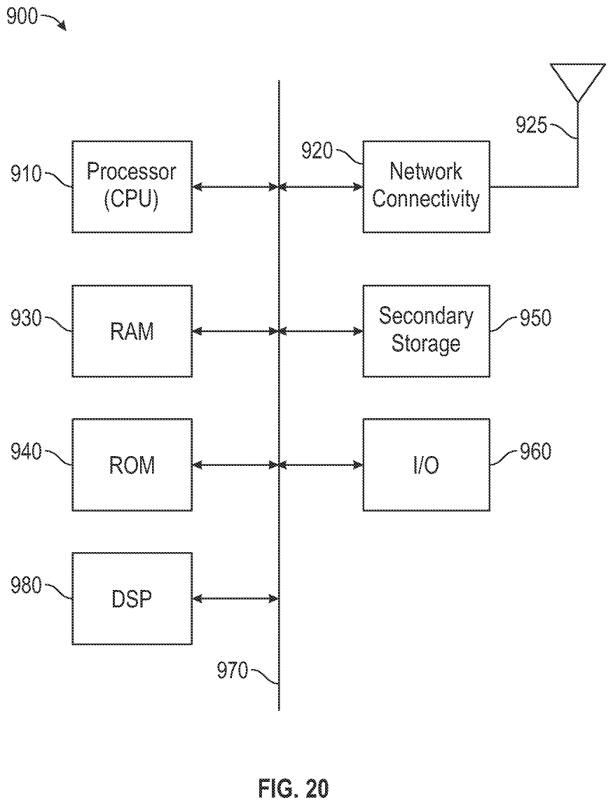

FIG. 20 is a diagram of an embodiment of a system 900 that includes a processor 910 suitable for implementing one or more embodiments disclosed herein, e.g., the gas analysis system 100 and/or gas analyzer 103. The processor 910 may control the overall operation of the system 900. In some embodiments, processor 910 may be a microcontroller including components described below, for example, RAM 930, ROM 940, and/or I/O 960. Although only one processor is shown, multiple processors may be employed in practice.

In addition to the processor 910 (which may be referred to as a central processor unit or CPU), of the system 900 might include network connectivity devices 920, random access memory (RAM) 930, read only memory (ROM) 940, secondary storage 950, and input/output (I/O) devices 960. These components might communicate with one another via a bus 970. In some cases, some of these components may not be present or may be combined in various combinations with one another or with other components not shown. These components might be located in a single physical entity or in more than one physical entity. Any actions described herein as being taken by the processor 910 might be taken by the processor 910 alone or by the processor 910 in conjunction with one or more components shown or not shown in the drawing, such as a digital signal processor (DSP) 980. Although the DSP 980 is shown as a separate component, the DSP 980 might be incorporated into the processor 910.

The processor 910 executes instructions, codes, computer programs, or scripts that it might access from the network connectivity devices 920, RAM 930, ROM 940, or secondary storage 950 (which might include various disk-based systems such as hard disk,, or optical disk). While only one CPU 910 is shown, multiple processors may be present. Thus, while instructions may be discussed as being executed by a processor, the instructions may be executed simultaneously, serially, or otherwise by one or multiple processors. The processor 910 may be implemented as one or more CPU chips and may be a hardware device capable of executing computer instructions.

The network connectivity devices 920 may take the form of modems, modem banks, Ethernet devices, universal serial bus (USB) interface devices, serial interfaces, token ring devices, fiber distributed data interface (FDDI) devices, wireless local area network (WLAN) devices, radio transceiver devices such as code division multiple access (CDMA) devices, global system for mobile communications (GSM) radio transceiver devices, universal mobile telecommunications system (UMTS) radio transceiver devices, long term evolution (LTE) radio transceiver devices, worldwide interoperability for microwave access (WiMAX) devices, controller area network (CAN), domestic digital bus (D2B), and/or other well-known devices for connecting to networks. These network connectivity devices 920 may enable the processor 910 to communicate with the Internet or one or more telecommunications networks or other networks from which the processor 910 might receive information or to which the processor 910 might output information. The network connectivity devices 920 might also include one or more transceiver components 925 capable of transmitting and/or receiving data wirelessly.

The RAM 930 might be used to store volatile data and perhaps to store instructions that are executed by the processor 910. The ROM 940 is a non-volatile memory device that typically has a smaller memory capacity than the memory capacity of the secondary storage 950. ROM 940 might be used to store instructions and perhaps data that are read during execution of the instructions. Access to both RAM 930 and ROM 940 is typically faster than to secondary storage 950. The secondary storage 950 is typically comprised of one or more disk drives or tape drives and might be used for non-volatile storage of data or as an over-flow data storage device if RAM 930 is not large enough to hold all working data. Secondary storage 950 may be used to store programs that are loaded into RAM 930 when such programs are selected for execution.

The I/O devices 960 may include liquid crystal displays (LCDs), touch screen displays, keyboards, keypads, switches, dials, mice, track balls, voice recognizers, card readers, paper tape readers, printers, video monitors, electrical connectors, electrical excitation sources, electrical measurement devices, switching devices, relay devices, or other well-known input/output devices. Also, the transceiver component 925 might be considered to be a component of the I/O devices 960 instead of or in addition to being a component of the network connectivity devices 920.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

What is claimed is:

1. A gas analysis system, comprising:
a gas extraction assembly comprising:
an extraction chamber having an upper end, a lower end, and an interior space;
one or more retainers positioned within the interior space of the extraction chamber; and
gas permeable membrane retained within the interior space of the extraction chamber by the one or more retainers, the gas permeable membrane disposed in S-shaped curves extending from adjacent the upper end to adjacent the lower end within the interior space of the extraction chamber.

2. The gas analysis system of claim 1, further comprising:
a transformer comprising an electrical winding and a fluid system comprising insulating fluid; and
a manifold having first interface in fluid communication with the transformer, a second interface in fluid communication with the extraction chamber, and one or more channels to communicate the fluid between the first and second interfaces.

3. The gas analysis system of claim 2, wherein the first and second interfaces of the manifold each include a supply channel and a return channel.

4. The gas analysis system of claim 3, wherein the extraction chamber further comprises:
an outer cover defining the interior space of the extraction chamber; and
a supply conduit centrally located in the extraction chamber, the supply conduit in fluid communication with the supply channel of the second interface of the manifold at a first end adjacent the lower end of the chamber and an output port located adjacent the upper end of the extraction chamber, wherein the output port is in fluid communication with the interior space of the extraction chamber.

5. The gas analysis system of claim 4, wherein the each of the one or more retainers is further defined as an O-shaped ring having an outer circumference edge, an inner circumference edge defining an inner opening, an upper surface, a lower surface, and a plurality of guide openings extending from the upper surface through to the lower surface and located adjacent and between the outer and inner circumference edges, wherein the outer circumference edge is adjacent and frictionally engages an interior wall of the chamber and the inner circumference edge is adjacent and frictionally engages an outer wall of the supply conduit, and wherein the gas permeable material passes through the plurality of guide openings.

6. The gas analysis system of claim 5, wherein the gas permeable membrane is further defined as gas permeable tubing having a first end and a second end.

7. The gas analysis system of claim 6, further comprising:
a gas analyzer having one or more sensors for analyzing characteristics of gas extracted from the fluid, the gas analyzer in fluid communication with the first and second ends of the gas permeable tubing to receive the gas extracted from the fluid for analysis by the gas analyzer.

8. The gas analysis system of claim 2, further comprising one or more pumps in fluid communication with the manifold to promote the communication of fluid between the first and second interfaces.

9. The gas analysis system of claim 2, wherein the second interface of the manifold is located on an upper surface of the manifold, wherein the first interface of the manifold is located on a first side of the manifold, and wherein the first side of the manifold is angled at approximately 45 degrees relative to a horizontal plane of the upper surface.

10. A gas analysis system, comprising
a transformer having an electrical winding and a fluid system comprising insulating fluid;
a gas extraction assembly having an extraction chamber and gas permeable membrane retained within the extraction chamber;
a manifold having a first interface and a second interface, the second interface in fluid communication with the gas extraction assembly, and one or more channels to communicate the insulating fluid between the first and second interfaces; and
an attachment system comprising:
an adapter pipe having a first end, a second end, an elongate portion extending from the first end towards the second end, and a bend portion provided adjacent the elongate portion and extending to adjacent the second end,
a first interface adjacent the first end of the adapter pipe in fluid communication with the transformer, and
a second interface adjacent the second end of the adapter pipe in fluid communication with the first interface of the manifold.

11. The gas analysis system of claim 10, wherein the second interface of the attachment system includes an inlet port and an outlet port, the inlet port in fluid communication with an outlet port of the second interface of the manifold, the outlet port in fluid communication with an inlet port of the second interface of the manifold.

12. The gas analysis system of claim 11, wherein the attachment system further comprises an extension tube provided within the adapter pipe and attached to the outlet port of the second interface of the attachment system at a first end of the extension tube, and wherein the extension tube extends to a second end of the extension tube adjacent the first end of the adapter pipe.

13. The gas analysis system of claim 12, wherein the extension tube has an outer diameter that is less than an inner diameter of the adapter pipe, and wherein the extension tube is flexible plastic, rubber or metallic, or a combination thereof.

14. The gas analysis system of claim 10, where a horizontal plane extends between first and second sides of the manifold, and wherein the gas extraction assembly is mounted on an upper side of the manifold such that the extraction chamber extends vertically relative to the horizontal plane.

15. The gas analysis system of claim 14, wherein the first interface of the manifold is located on the first side of the manifold, and wherein the first interface is angled at approximately 45 degrees relative to the horizontal plane.

16. The gas analysis system of claim 15, wherein in a first position of the attachment system the elongate portion of the adapter pipe is oriented substantially parallel to the horizontal plane, and wherein in a second position of the attachment system the elongate portion of the adapter pipe is oriented substantially perpendicular to the horizontal plane, and wherein the bend portion of the adapter pipe bends such that the second interface adjacent the second end of the adapter pipe mates with the angled first interface of the manifold when the attachment system is in either the first or second positions.

17. The gas analysis system of claim 16, wherein when the attachment system is in the first or second positions, the manifold is positioned such that the horizontal plane is substantially parallel to an adjacent ground surface and the gas extraction assembly extends vertically relative to the horizontal plane and the ground surface.

18. The gas analysis system of claim 10, wherein the attachment system fluidly communicates with a fluid in the transformer via one or more of:
attachment of the first interface adjacent the first end of the adapter pipe to a drain valve of the transformer; or
attachment to a fluid access point of the transformer via one or more pipe or coupling pieces, pipe fittings, elbows in communication with the first interface adjacent the first end of the adapter pipe.

19. A method analyzing dissolved gases in a fluid of a transformer, the method comprising:
providing an adapter pipe having a first end and a second end, the first end in fluid communication with the fluid of the transformer, the second end in fluid communication with a gas analysis system, the adapter pipe having an extension tube provided within the adapter pipe and extending within the adapter pipe between the first end and the second end of the adapter pipe;
supplying the fluid through the extension tube from a first end of the extension tube adjacent the first end of the adapter pipe to a second end of the extension tube adjacent the second end of the adapter pipe, the second end of the extension tube coupled to communicate with a first port of the gas analysis system;
communicating the fluid from the second end of the extension tube, via the first port, into an extraction chamber of the gas analysis system, the extraction chamber having gas permeable membrane disposed therein;
obtaining the dissolved gas via the gas permeable membrane;
analyzing characteristics of the dissolved gas;
communicating the fluid from the extraction chamber to a second port of the gas analysis system; and
returning the fluid, via the second port, to the second end of the adapter pipe, the second port adjacent the first port such that the supply fluid is supplied adjacent the first end of the adapter pipe and return fluid is returned adjacent the second end of the adapter pipe.
